(19) United States
(12) Patent Application Publication
(10) Pub. No.: US 2005/0250799 A1
(43) Pub. Date: Nov. 10, 2005

(54) Protease inhibitors for treatment of wrinkles

(76) Inventors: Seishiro Fujii, Boston, MA (US); Antonis S. Zervos, Oviedo, FL (US); Satoshi Hirakawa, Arlington, MA (US); Michael Detmar, Boppelsen (CH)

Correspondence Address: FISH & RICHARDSON PC, P.O. BOX 1022, MINNEAPOLIS, MN, 55440-1022, US

(21) Appl. No.: 11/052,149

(22) Filed: Feb. 7, 2005

Related U.S. Application Data
(60) Provisional application No. 60/542,187, filed on Feb. 5, 2004.

(51) Int. Cl. A61K 31/515; A61K 7/42

(52) U.S. Cl. 514/270; 424/59

(57) ABSTRACT

Methods, compositions, and kits are provided for the use of protease inhibitors, such as Ucf-101, to reduce wrinkles or other skin damage.

### FEDERALLY SPONSORED RESEARCH OR DEVELOPMENT

The U.S. Government may have certain rights in this invention pursuant to Grant No. CA92644 to Dr. Michael Detmar, awarded by National Institutes of Health.

### CROSS-REFERENCE TO RELATED APPLICATIONS

This application claims priority to U.S. Application Ser. No. 60/542,187, filed on Feb. 5, 2004, the contents of which are incorporated by reference in their entirety.

### BACKGROUND

Proteases and their inhibitors are involved in regulating a multitude of biological functions. Several proteases are involved in apoptosis. Caspases are cysteine proteases that cleave other cellular proteins (including other caspases) after an aspartic acid residue, and are crucial for the apoptotic process. Omi/HtrA2 is a mitochondrial serine protease that is released into the cytosol during apoptosis and promotes cytochrome c (Cyt c) dependent caspase activation by neutralizing inhibitor of apoptosis proteins (IAPs) via its IAP-binding motif. The protease activity of Omi/HtrA2 also contributes to the progression of both apoptosis and caspase-independent cell death. Other proteins are also involved in apoptosis.

### SUMMARY OF THE INVENTION

The invention includes, inter alia, methods and compositions (e.g., cosmetic or therapeutic methods and compositions) useful in treating skin, e.g., preventing or reducing skin damage or another skin condition. For example, the methods and compositions can be used to ameliorate symptoms of UVB-induced photodamage (e.g., chronic photodamage) and wrinkles. The inventors have found, for example, that wrinkles can be prevented and/or reduced by administering to a subject, e.g., a human, a protease inhibitor.

The protease inhibitor is preferably an inhibitor of a protease activated in, or involved in, apoptosis, e.g., a caspase (e.g., caspase-7 or caspase-9) or a serine protease involved in apoptosis, e.g., a mammalian serine protease having statistically significant sequence homology to bacterial HtrA chaperones. For example, the protease inhibitor can have an IC50 of less than 80, 50, 20, or 10 μM for the protease. The protease inhibitor can preferentially inhibit the protease activated in, or involved in, apoptosis, by an IC50 that is at least 5, 10, or 20 fold better (i.e., less) than its IC50 for another mammalian protease, e.g., human kallikrein, human plasmin, or human thrombin.

In a preferred embodiment, the protease inhibitor is a caspase inhibitor.

In a preferred embodiment, the protease inhibitor is an inhibitor of the mitochondrial serine protease Omi/HtrA2.

In a preferred embodiment, the protease inhibitor is a cell-permeable agent.

In a preferred embodiment, the protease inhibitor has following formula (I): 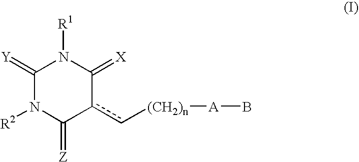
wherein:
each of R¹ and R² is, independently, aryl, heteroaryl, cycloalkyl, heterocycloalkyl, cycloalkenyl, heterocycloalkenyl, aralkyl, or heteroaralkyl and is optionally substituted with 1-5 R³;
each of X, Y, and Z is, independently, O or S;
- - - represents an optional double bond;
n is 0-20;
each of A and B is, independently, aryl or heteroaryl, and is optionally substituted with 1-5 R³; and
each R³ is, independently, halo, hydroxy, C₁-C₁₀ alkyl, C₁-C₁₀ hydroxyalkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, C₁-C₆ alkylanhydrido, or C₁-C₆ hydroxycarbonylalkyl.

In a preferred embodiment, R¹ and R² are both aryl.

In a preferred embodiment, R¹ and R² are both phenyl.

In a preferred embodiment, one of X, Y, and Z is S, and the other two are 0. In another embodiment, Y is S and X and Z are O.

In a preferred embodiment, the double bond is present.

In a preferred embodiment n is 0.

In a preferred embodiment one of A and B is heteroaryl, and the other is aryl.

In a preferred embodiment A is heteroaryl and Bis aryl.

In a preferred embodiment, A has formula (II): 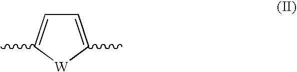
wherein,
W is O, S, or NR^{a}; and
R^{a} is hydrogen or C₁-C₆ alkyl. Preferably, W is O.

In a preferred embodiment, B has formula (III): 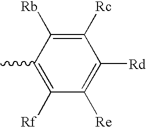
wherein, each of R^{b}, R^{c}, R^{d}, Re, and R^{f} is, independently, hydrogen, halo, hydroxy, C₁-C₁₀ alkyl, C₁-C₁₀ hydroxyalkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, C₁-C₆ alkylanhydrido, or C₁-C₆ hydroxycarbonylalkyl.

In a preferred embodiment, each of R^{b}, R^{c}, R^{d}, Re, and R^{f} is, independently, hydrogen, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

In a preferred embodiment each of R^{c}, R^{d}, and R^{e} is hydrogen and each of R^{b} and R^{f} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

In a preferred embodiment, one of R^{b} or R^{f} is nitro.

In a preferred embodiment, each of R^{b}, R^{c}, and R^{f} is hydrogen, and each of R^{d} and R^{e} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

In a preferred embodiment, one of R^{d} and R^{e} is halo, and the other is C₁-C₄ alkoxy.

In a preferred embodiment, R^{d} is OCH₃ and R^{e} is C1.

In a preferred embodiment, each of R^{b}, R^{d}, R^{e} and R^{f} is hydrogen, and R^{c} is hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

In a preferred embodiment, R^{c} is carboxy.

In a preferred embodiment, each of R^{c}, R^{d}, and R^{f} is hydrogen, and each of R^{b} and R^{e} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

In a preferred embodiment, one of R^{b} and R^{e} is nitro.

A preferred inhibitor is selected from:

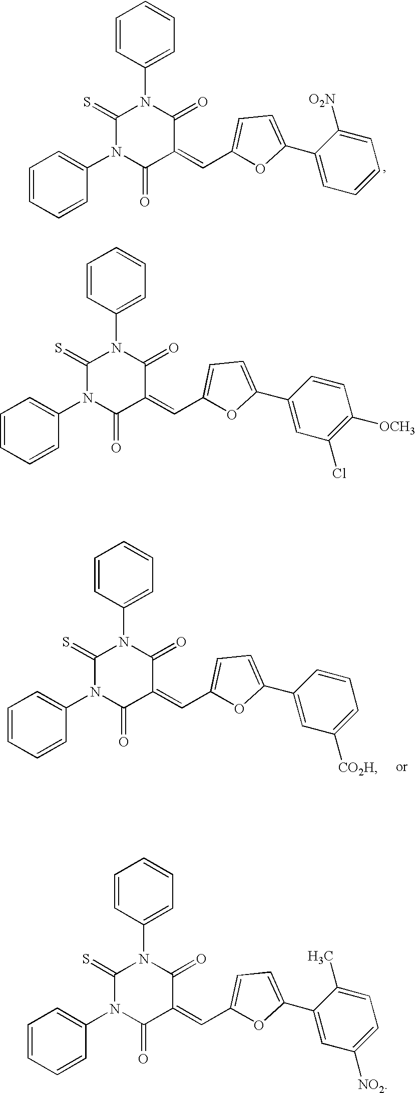

The protease inhibitor can be administered topically, e.g., to the face, chest, neck, hands, and other regions of the body in need of skin treatment, e.g., wrinkle treatment. The treatment can involve more than one administration, e.g., at least two, three, or four administrations, of the protease inhibitor. The treatment can, e.g., involve daily administration of the protease inhibitor (e.g., once a day or twice a day) for a specified number of days, e.g., at least 2 days, 3 days, 4 days, 7 days, 14 days, 21 days, one month, three months, six months or longer.

In preferred embodiments, wrinkles are reduced in the subject by at least 5%, preferably at least 10%, more preferably at least 20%, 25% or more (e.g., using a qualitative or quantitative method of wrinkle evaluation described herein).

In one embodiment, the subject's skin has been chronically exposed to UV radiation, e.g., the subject has been exposed to the sun repeatedly (more than once) over a period of at least one week, or the subject shows symptoms of damaged or aging skin, e.g., wrinkles.

In another embodiment, the method includes: identifying a subject in need of preventing or treating wrinkle formation or other skin condition: administering the protease inhibitor, e.g., a protease inhibitor described herein; and evaluating the effect of the administration on wrinkle formation, other skin condition, or on apoptosis, e.g., in the skin of the subject, e.g., at the site of application. In a preferred embodiment, the subject's skin has been exposed to UV, e.g., UVB radiation. The identification of a subject in need of preventing or reducing wrinkles or other skin condition can be performed e.g., by the subject, by a health care provider, or by a provider of cosmetics. The protease inhibitor may be administered, e.g., by the subject, by a health care provider, or by a provider of cosmetics. Likewise, the evaluation of the effect on wrinkle formation may be performed, e.g., by the subject, by a health care provider, or by a provider of cosmetics.

In one embodiment, the protease inhibitor is provided in a cosmetically acceptable composition, e.g., a cream, lotion, foam, gel, or other cosmetic preparation. In another embodiment, the protease inhibitor can be provided in a pharmaceutically acceptable carrier, e.g., a sterile buffer.

In one embodiment, the protease inhibitor is administered in combination with a second agent, e.g., a cosmetic agent, e.g., one or more of: a fragrance, a tanning agent, a moisturizer, an essential fatty acid, ceramide, an essential oil, a sunscreen agent (e.g., octyl methoxycinnamate, aminobenzoic acid, oxybenzone, padimate O, homosalate, or titanium dioxide), a protein or protein hydrolysate, an amino acid, a vitamin (e.g., retinol/vitamin A and its derivatives or tocopherol/vitamin E), a polyol, urea, allantoin, a sugar or sugar derivative, a botanical extract (e.g., an extract of Paraguay tea, Kola, Guarana, or aloe vera).

The protease inhibitor can be effective to reduce the appearance of wrinkles when applied to the skin, e.g., for a period of at least 2 to 100 days, more preferably at least 7 to 90 days, even more preferably 14 to 60 days, or it can be effective to reduce the appearance of wrinkles for a longer term, e.g., at least 3 to 9 months, more preferably 4 to 8 months, or about 6 months.

In some embodiments, the protease inhibitor can be modified, e.g., derivatized or conjugated to another molecule. In preferred embodiments, the protease inhibitor is modified to make it more suitable for human use, e.g., to make the protease inhibitor more active, more stable, or more soluble.

In another aspect, the disclosure features a method of providing wrinkle protection to a subject by supplying a protease inhibitor, e.g., a protease inhibitor described herein, e.g., Ucf-101, to the subject, preferably with instructions to apply prior to, or after, UV exposure, e.g., UVB, e.g., sunlight exposure.

In still another aspect, the disclosure features a method of providing wrinkle protection or modulating wrinkle formation to a subject by administering an inhibitor of apoptosis, e.g., an inhibitor of apoptosis in fibroblasts. The inhibitor can be applied topically, e.g., to the skin, e.g., at a site of wrinkling or skin damage, e.g., a cut, blister, scarring, UV-B damage, a burn, and so forth. In one embodiment, the inhibitor is a protease inhibitor, e.g., an inhibitor of a protease activated in, or involved in, apoptosis, e.g., a caspase (e.g., caspase-7 or caspase-9) or a serine protease involved in apoptosis, e.g., a mammalian serine protease having statistically significant sequence homology to bacterial HtrA chaperones.

In another aspect, the disclosure features a kit for providing wrinkle protection to a subject. The kit includes a composition containing a protease inhibitor, e.g., a protease inhibitor described herein, e.g., Ucf-101; and instructions for using the protease inhibitor to prevent, treat or reduce wrinkles. The instructions can include instructions to apply the composition prior to, or after, UV, e.g., UVB exposure, e.g., sunlight exposure.

In a preferred embodiment, the weight percent of the protease inhibitor in the composition ranges from 0.01% to 10%, e.g., 0.01% to 3%, 3% to 6%, or 6% to 10%. In another preferred embodiment, the weight percent of the protease inhibitor ranges from 0.05% to 10%, e.g., 0.05% to 5%, e.g., 0.05% to 3%. In a preferred embodiment, the composition also has a fragrance, a preservative, or other cosmetic ingredient, e.g., a moisturizer, or sunscreen agent, e.g., octyl methoxycinnamate, aminobenzoic acid, oxybenzone, padimate 0, homosalate, or titanium dioxide.

Unless otherwise defined, all technical and scientific terms used herein have the same meaning as commonly understood by one df ordinary skill in the art. All publications mentioned herein are incorporated by reference in their entirety. In case of conflict, the present specification, including definitions, will control. In addition, the materials, methods, and examples are illustrative only and not intended to be limiting.

Other features or advantages of the present inventions will be apparent from the following detailed description of several embodiments, and also from the appending claims.

### DETAILED DESCRIPTION

The invention relates to, inter alia, methods for preventing or reducing wrinkle formation or other skin condition (e.g., skin damage) by administering a protease inhibitor to a subject. A preferred protease inhibitor is an inhibitor of a protease activated in, or involved in, apoptosis, for example a serine protease involved in apoptosis, or a caspase. For example, the protease inhibitor is an inhibitor the mitochondrial serine protease Omi/HtrA2. An exemplary inhibitor is Ucf-101.

Protease Inhibitors

Caspase inhibitors act by binding to the active site of caspases and form either a reversible or an irreversible linkage. A caspase inhibitor can include a peptide recognition sequence attached to a functional group such as an aldehyde (CHO), chloromethyl-ketone (CMK), fluoromethylketone (FMK) or fluoroacyloxymethyl ketone (FAOM). Caspase inhibitors with a CHO group are typically reversible and those with a CMK, FMK, or FAOM group are typically irreversible. FMK exhibits slightly less reactivity than CMK and therefore is more specific for the enzyme site being inhibited. The peptide recognition sequence corresponding to that found in endogenous substrates determines the specificity of a particular caspase. Peptides with the Ac-YVAD-CHO sequence are potent inhibitors of caspases 1 and 4 (Kᵢ≈10 nM), and exhibit very weak inhibitory effect on caspases 3 and 7 (Kᵢ ≧50 μM). Exclusion of the tyrosine residue from the inhibitor peptide results in a potent but less specific inhibitor, for example, Z-VAD-FMK inhibits not only caspases 1 and 4, but also caspases 3 and 7. Inhibitors with the recognition sequence DEVD are potent inhibitors of caspase-3 (Kᵢ=0.5 nM). They also inhibit caspases 3, 6, 7, 8, and 10 at much higher concentrations. To increase the cell permeability of a caspase inhibitor, aspartic acid residues can be esterified (OMe). In some embodiment, a peptide corresponding to the hydrophobic region of Kaposi fibroblast growth factor or another cell permeability enhancing agent can be added to the recognition sequence to increase cell permeability.

A caspase inhibitor can also be an inhibitor of apoptosis protein (IAP), e.g., a protein that includes at least one baculovirus inhibitor of apoptosis protein repeat (BIR) domain (Pfam accession number: PF00653 (Bateman et al. (2004) Nucleic Acids Res. 32:D138-41)). A caspase inhibitor can also be a functional fragment or derivative of a protein that can inhibit a caspase, e.g., fragment that retains inhibitor function.

Ucf-101

Ucf-101 is cell-permeable furfurylidine-thiobarbituric acid compound that acts as a potent, specific, competitive and reversible inhibitor of the pro-apoptotic, heat-inducible, mitochondrial serine protease Omi/HtrA2 (IC₅₀=9.5 μM for His-Omi 134-458). See Cilenti et al. (2003) J. Biol. Chem. 278 (13):11489-94. Ucf-101 shows very little activity against various other serine proteases tested (IC₅₀=200 μM). Ucf-101 is reported to block Omi/HtrA2 induced cell death in caspase-9 (−/−) null fibroblasts. Ucf-101 is commercially available, e.g., from Calbiochem. Because it is a small molecule, Ucf-101 can enter mammalian cells to exert its action. Besides Ucf-101, structurally related and other compounds that inhibit Omi/HtrA2, e.g., a compound described herein, can be used in the methods described herein.

Assays and techniques for working with proteases and their inhibitors are routine in the art and can be found, e.g., in Proteolytic enzymes: serine and cysteine peptidases, Methods in Enzymology Vol 244, A. J. Barrett, Editor (Academic Press 1995). Omi protease activity is described, e.g., in Faccio (2000) J Biol. Chem. 275 (4): 2581-8. Additional methods for evaluating inhibitors of Omi are described, e.g., in Cilenti et al. (2003) J. Biol. Chem. 278 (13):11489-94

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂— (methylene), —CH₂CH₂— (ethylene), and —CH₂CH₂CH₂— (propylene).

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The terms “alkylamino” and “dialkylamino” refer to —NH(alkyl) and —NH(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system. Any ring atom can be substituted. Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted. The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3,1-6, or 1-9 heteroatoms of N, O, or S if monocyclic, bicyclic, or tricyclic, respectively). The heteroatom may optionally be the point of attachment of the heterocyclyl substituent. Any ring atom can be substituted. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted. The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3,1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted. The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3,1-6, or 1-9 heteroatoms of N, O, or S if monocyclic, bicyclic, or tricyclic, respectively). Any ring atom can be substituted. Examples of heteroaryl groups include but are not limited to furanyl, thienyl, pyrrolyl, pyridinyl.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The terms “aminocarbonyl,” “alkoxycarbonyl,” “alkylanhydrido,” or “hydroxycarbonylalkyl” refer to the radicals —C(O)NH₂, —C(O)O(alkyl), —C(O)OC(O)(alkyl), and (alkyl)CO₂H, respectively.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, written and graphical descriptions are made in terms of what the art recognizes as the predominant resonance form for a particular species.

Combinations of substituents and variables typically refer to those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The compounds described herein can be obtained from commercial sources (e.g., a commercially available compound library) or synthesized by conventional methods as shown below using commercially available starting materials and reagents.

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein may contain one or more asymmetric centers and thus occur as racemates and racemic mixtures, single enantiomers, individual diastereomers and diastereomeric mixtures. All such isomeric forms of these compounds are expressly included. The compounds may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included. The compounds may also be represented in multiple tautomeric forms, in such instances, the disclosure expressly includes all tautomeric forms of the compounds described herein, even though only a single tautomeric form may be represented (e.g., alkylation of a ring system may result in alkylation at multiple sites, the disclosure expressly includes all such reaction products). All such isomeric forms of such compounds are expressly included. All crystal forms of the compounds described herein are expressly included.

The compounds may include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Wrinkles

Wrinkles are generally a result of the natural aging process of the skin and of exposure to the sun's ultraviolet rays. A wrinkle is a configuration change in the surface of the skin, without specific structural alterations at the histological level. Generally, wrinkles are classified as described in Kligman et al. (1985) Br J Derm 113:37-42, herein incorporated by reference. Kligman classifies wrinkles into three classes: linear wrinkles, glyphic wrinkles, and crinkles. Linear wrinkles are straight, found generally in the facial skin, and are caused by natural aging or exposure to ultraviolet light. Glyphic wrinkles are shaped as apparent triangles or rectangles of wrinkles, are found on the face, hands, and neck exposed to sunlight, and are aggravated by exposure to ultraviolet light or dermatoheliosis. Crinkles are thin, crinkled wrinkles on flabby skin, found anywhere on the skin, but typically on the backs of hands and around the eyelids.

Linear wrinkles can be further subclassified into (a) regular wrinkles and (b) fine wrinkles. Regular wrinkles are long, deep, clear, and are also referred to as crow's feet. Fine wrinkles are thin and shallow. Regular wrinkles have a width of at least about 155 microns (0-32 Hz), preferably about 160 to 250 microns. Fine wrinkles have a width of less than about 154 microns, preferably about 40 to 154 microns (32-126 Hz), as calculated e.g., in a power spectrum obtained through transforming three dimensional shape data into data in a frequency domain by two-dimensional Fourier transformation (using, e.g., the Shiseido Wrinkle Analyzer 3D Pro system, essentially as described in Takasu et al. (1996) J Soc Cosmet Chem Japan 29:394-405; and Japanese Published Patent Application No. 07-113623, published May 2, 1995).

An effective amount of a composition is defined as the amount of the composition which, upon administration to a subject, prevents the formation of wrinkles, or fine wrinkles, in the subject, or reduces the appearance of wrinkles, or fine wrinkles, in the subject. The effective amount to be administered to a subject is typically based on a variety of factors including age, sex, surface area, weight, and conditions of the skin. Body surface area may be approximately determined from height and weight of the patient. See, e.g., Scientific Tables, Geigy Pharmaceuticals, Ardley, New York, 1970, 537. Effective doses will vary, as recognized by those skilled in the art, dependant on route of administration, excipient usage, and the possibility of co-usage with other treatments such as usage of other wrinkle reducing compounds.

As used herein, “preventing or treating a wrinkle” means the application or administration of a therapeutic agent to a subject who has a wrinkle or has a predisposition toward wrinkles, or has been exposed to an agent likely to cause wrinkles, e.g., UV radiation, e.g., UVB irradiation, with the purpose to reduce, improve, alleviate, alter, remedy, ameliorate, or affect, the appearance of the wrinkle or the formation of the wrinkle. The therapeutic agent can be administered to the subject by the subject himself or herself, or by another person, e.g., a health care provider or a provider of cosmetics. In preferred embodiments of the methods described herein, wrinkles are reduced in the subject by at least 5%, preferably at least 10%, more preferably at least 20%, 25% or more.

The methods and compositions can be used prophylactically or they can be used to prevent further wrinkle formation or reduce the appearance of wrinkles in a subject. The composition can also be used for the manufacture of a medicament or cosmetic for preventing, reducing or treating wrinkles.

Administration of Protease Inhibitor Compositions

The pharmaceutical composition for the prevention or reduction of wrinkles or other skin condition may be administered via the parenteral route, including orally, topically, subcutaneously, intraperitoneally, intramuscularly, intranasally, and intravenously. Topical administration is preferred. Repeated administration of the composition, e.g., repeated topical administration, can be used. More than one route of administration can be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the wrinkle reducing composition.

A composition described herein can also be formulated into dosage forms for other routes of administration utilizing conventional methods. A pharmaceutical composition can be formulated, for example, in dosage forms for oral administration in a capsule, a tablet (each including timed release and sustained release formulations), or a gel seal. Capsules may comprise any standard pharmaceutically acceptable material such as gelatin or cellulose derivatives. Tablets may be formulated in accordance with the conventional procedure by compressing mixtures of a protease inhibitor compound and a solid carrier, and a lubricant. Examples of solid carriers include starch and sugar bentonite. The wrinkle reducing composition can also be administered in a form of a hard shell tablet or capsule containing, for example, lactose or mannitol as a binder and a conventional filler and a tableting agent.

Topical administration of the wrinkle reducing compounds described herein presents a preferred route of administration amongst the many different routes described above. For topical application, the composition can include a medium compatible with skin. Such topical pharmaceutical compositions can exist in many forms, e.g., in the form of a solution, cream, ointment, gel, lotion, shampoo, or aerosol formulation adapted for application to the skin. The weight percent of the active ingredient in the composition, e.g., the protease inhibitor compound, useful in preventing or reducing wrinkles ranges from 0.01% to 10% (based on the total weight of the composition) in admixture with a pharmaceutically acceptable carrier. A wide variety of carrier materials can be employed in the wrinkle reducing composition described herein such as alcohols, aloe vera gel, allantoin, glycerine, vitamin A and E oils, mineral oils, and polyethylene glycols. Other additives, e.g., preservatives, fragrance, sunscreen, or other cosmetic ingredients, can be present in the composition. The topical composition can be applied and removed immediately, or it can be applied and left on the skin surface, e.g., the face, for an extended period of time, e.g., overnight or throughout the day.

Measurement of Wrinkles

The effect of a compound on the formation or appearance of wrinkles can be evaluated qualitatively, e.g., by visual inspection, or quantitatively, e.g., by computer assisted measurements of wrinkle morphology. Preferably, wrinkle morphology is quantitatively analyzed. Examples of quantitative methods for measuring wrinkles include, but are not limited to, the optical cut technique employing a laser beam, as proposed by Hoshino (1992) Pixel 45:121, herein incorporated by reference; or methods which analyze three-dimensional skin replicas, e.g., the Shiseido Wrinkle Analyzer 3D Pro system (Takasu et al. (1996) J Soc Cosmet Chem Japan 29:394-405; Japanese Published Patent Application No. 07-113623, published May 2, 1995 (corresponds to U.S. patent application Ser. No. 08/364,346)). The SILFLO® (Flexico Development Ltd.) system or a similar system can be used to take a replica of the skin. Irregularities on the surface of the skin replica, i.e., wrinkles, are analyzed, e.g., with the Shiseido Wrinkle Analyzer 3D Pro or a similar system, to provide three-dimensional shape data from the heights at points on a two-dimensional plane corresponding to the skin. According to the three-dimensional data, the length, width, depth, area, and volume of each wrinkle is calculated. According to the parameters for regular and fine wrinkles described herein, different classes of wrinkles, including the subclasses of regular and fine wrinkles, can thus be individually recognized and scored.

Kits

The protease inhibitor described herein can be provided in a kit. The kit includes (a) the inhibitor, e.g., a composition that includes the inhibitor, and (b) informational material. The informational material can be descriptive, instructional, marketing or other material that relates to the methods described herein and/or the use of the inhibitor for the methods described herein. For example, the informational material relates to wrinkles or other forms of photodamage.

In one embodiment, the informational material can include instructions to administer the inhibitor in a suitable manner to perform the methods described herein, e.g., in a suitable dose, dosage form, or mode of administration (e.g., a dose, dosage form, or mode of administration described herein). A preferred dose is between 0.05 and 10% of the inhibitor; a preferred mode of administration is topical. In another embodiment, the informational material can include instructions to administer the inhibitor to a suitable subject, e.g., a human, e.g., a human having, or at risk for, UVB induced wrinkles. For example, the material can include instructions to administer the inhibitor to the face, hands, or neck.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about the inhibitor and/or its use in the methods described herein. Of course, the informational material can also be provided in any combination of formats.

In addition to the inhibitor, the composition of the kit can include other ingredients, such as a solvent or buffer, a stabilizer, a preservative, a fragrance or other cosmetic ingredient, and/or a second agent for treating a condition or disorder described herein, e.g., a sunscreen. Alternatively, the other ingredients can be included in the kit, but in different compositions or containers than the inhibitor. In such embodiments, the kit can include instructions for admixing the inhibitor and the other ingredients, or for using the inhibitor together with the other ingredients.

The inhibitor can be provided in any form, e.g., liquid, dried or lyophilized form. It is preferred that the inhibitor be substantially pure and/or sterile. When the inhibitor is provided in a liquid solution, the liquid solution preferably is an aqueous solution, with a sterile aqueous solution being preferred. When the inhibitor is provided as a dried form, reconstitution generally is by the addition of a suitable solvent. The solvent, e.g., sterile water or buffer, can optionally be provided in the kit.

The kit can include one or more containers for the composition containing the inhibitor. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the inhibitor. For example, the kit includes a plurality of syringes, ampules, foil packets, or blister packs, each containing a single unit dose of the inhibitor. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a swab.

The following specific examples are to be construed as merely illustrative, and not limiting of the remainder of the disclosure in any way whatsoever.

### EXAMPLES

### Example 1

### Ucf-101 Effect on Ear Thickness

This example shows that ucf-101 is not irritating to the skin.

Control thickness of mice ears (FVB strain, male, 7 weeks of age, n=3/group) was measured with a thickness gauge (Mitsutoyo Corp.). After measuring the thickness of both ears, 10 μl of ucf-101 solution (1.0% or 0.1%) was applied to the left ear and 10 μl of solvent was applied to the right ear. Ear thickness was measured for three consecutive days after administration. The results (mean±S.D.) of both left and right ear thickness are provided in Table 1.

TABLE 1

Day

| | | | |
|---|---|---|---|
| 0 | 1 | 2 | 3 |

LEFT EAR

| | | | | |
|---|---|---|---|---|
| Group I | 28.0 | 28.7 | 28.7 | 28.3 |
| 1% Ucf-101 in DMSO | | | | |
| (×0.01 mm) | | | | |
| SD | 1.00 | 0.58 | 0.58 | 0.58 |
| Group II | 29.0 | 30.0 | 29.3 | 29.0 |
| 0.1% Ucf-101 in acetone | | | | |
| (×0.01 mm) | | | | |
| SD | 0.00 | 1.00 | 0.58 | 0.00 |

RIGHT EAR

| | | | | |
|---|---|---|---|---|
| Group I | 29.0 | 29.0 | 28.7 | 29.0 |
| DMSO | | | | |
| (×0.01 mm) | | | | |
| SD | 1.00 | 1.00 | 0.58 | 0.00 |
| Group II | 28.3 | 29.0 | 29.0 | 29.0 |
| acetone | | | | |
| (×0.01 mm) | | | | |
| SD | 1.16 | 1.00 | 0.00 | 0.00 |

As can be seen from Table 1, ucf-101 did not increase ear thickness when compared to solvent alone (DMSO and acetone). Thus, ucf-101 was not an irritant at a concentration of 1% in DMSO or 0.1% in acetone.

### Example 2

### Ucf-101 Effect on Ear Thickness on UVB-Exposed Skin

This example shows that ucf-101 is not irritating to UVB-exposed skin.

The ear thickness of mice (Swiss Webster strain, male, 7 weeks of age, n=3/group) was measured with a thickness gauge (Mitsutoyo Corp.) on day 0. After measuring the thickness of both ears, both ears were UVB-irradiated (80 mJ/cm²). 5 μl of 1% ucf-101 in DMSO was applied to the left ear and 5 μl of DMSO was applied to the right ear. After 24 hours (day 1), ear thickness was measured and a second dose administered (left ear 1% ucf-101 in DMSO; right ear: DMSO). After a second 24 hours (day 2), ear thickness was again measured and a third dose administered (left ear 1% ucf-101 in DMSO; right ear: DMSO). Ear thickness was then monitored over four additional consecutive days (days 3-6). The results (mean±S.D.) are provided in Table 2.

TABLE 2

| Day | | | | | | |
|---|---|---|---|---|---|---|
| 0 | 1 | 2 | 3 | 4 | 5 | 6 |

Left ear

| | | | | | | | |
|---|---|---|---|---|---|---|---|
| 1% Ucf-101 in | 31.0 | 36.3 | 40.0 | 42.3 | 42.7 | 39.3 | 38.3 |
| DMSO | | | | | | | |
| (×0.01 mm) | | | | | | | |
| SD | 1.00 | 2.08 | 1.00 | 1.53 | 5.51 | 4.04 | 2.31 |

Right ear

| | | | | | | | |
|---|---|---|---|---|---|---|---|
| DMSO | 31.0 | 37.0 | 39.3 | 42.3 | 42.7 | 40.0 | 38.3 |
| (×0.01 mm) | | | | | | | |
| SD | 1.00 | 3.46 | 0.58 | 1.53 | 5.51 | 5.20 | 2.31 |

As seen in Table 2, ucf-101 did not increase ear thickness (i.e., irritancy) when compared to control.

### Example 3

### Effect of Omi Inhibition on Wrinkles and Vessel Formation

This example shows that inhibition of Omi/HtrA2 reduces UVB-induced skin damage.

Five groups of mice (7-week-old female hairless Skh-1 mice, n=3/group) were used: group 1: UVB+1% ucf-101 in DMSO; group 2: UVB+DMSO control; group 3: UVB+0.1% ucf-101 in acetone; group 4: UVB+acetone control; group 5: no UVB control.

Groups 1, 2, 3 and 4 were exposed to UVB irradiation using fluorescent lamps (Southern New England Ultraviolet) to deliver 0.35 mW/cm² at the dorsal skin surface of the mice. After irradiation, the indicated samples (100 μl each) for each group were applied to the back skin. This procedure was repeated three times weekly for ten weeks, to simulate chronic UVB exposure. UVB irradiation began with a dose of 0.5 minimum erythema dose (MED) (20 mJ/cm²) and gradually increased in increments of 0.5 MED each day to a maximum dose of 4.5 MED. The total cumulative dose was 6.54 J/cm² UVB.

After 10 weeks, skin wrinkling was evaluated using five categories (0: no wrinkle; 1: slight wrinkle; 2: clear wrinkle; 3: strong wrinkle; 4: severe wrinkle). Mice of group 1 and group 2 were sacrificed and back skin samples were snap-frozen in liquid nitrogen. Immunohistochemical staining was performed on 7-micrometer frozen sections using a monoclonal rat anti-mouse CD31 antibody (BD Pharmingen). Representative sections were obtained from UVB irradiated mice and were analyzed using a Nikon E-600 microscope. Images were captured with a Spot digital camera (Diagnostic Instruments), and morphometric analyses were performed using the IP-LAB software. Areas occupied by blood vessels were determined in the dermis.

The results (mean±S.D.) are shown in Table 3 (wrinkling) and Table 4 (vessel area).

TABLE 3

| Group | wrinkle category (mean) | S.D. |
|---|---|---|
| 1 | 1.33 | 0.58 |
| 2 | 3.00 | 0.00 |
| 3 | 1.67 | 0.58 |
| 4 | 2.00 | 1.00 |
| 5 | 0.00 | 0.00 |

As can be seen by Table 3, inhibition of Omi/HtrA2 was effective at preventing and reducing UVB-induced wrinkle formation.

TABLE 4

| Group | Percent of vessel area (mean) | S.D. |
|---|---|---|
| 1 | 7.1% | 0.73 |
| 2 | 11.9% | 1.90 |
| 5 | 4.1% | 0.22 |

As can be seen by Table 4, inhibition of Omi/HtrA2 was effective at reducing UVB-induced vessel dilation.

### Example 4

### Effects of Test Samples on Ear Thickness

This example shows that test samples including ucf-101 are not irritating to the skin.

Control thickness of mice ears (FVB strain, female, 7 weeks of age, n=3/group) was measured with a thickness gauge (Mitsutoyo Corp.) on day 0. After measuring the thickness of both ears, 10 μl of test sample solution was applied to the left ear and 10 μl of solvent was applied to the right ear. After 24 hours (day 1), ear thickness was measured and a second dose administered (left ear: test solution, right ear: solvent). After a second 24 hours (day 2), ear thickness was measured and a third dose administered (left ear: test solution, right ear: solvent). After a third 24 hours (day 3), ear thickness was measured. The results (mean±S.D.) are provided in Table 5.

TABLE 5
LEFT EAR

| | Day | | | |
|---|---|---|---|---|
| | 0 | 1 | 2 | 3 |
| Group I | 25.7 | 26.3 | 27.3 | 28.3 |
| 0.1% Ucf-101 in 25% DMSO + | | | | |
| 75% ethanol | | | | |
| (×0.01 mm) | | | | |
| S.D. | 0.58 | 0.58 | 0.58 | 1.53 |
| Group II | 26.3 | 27.7 | 26.3 | 27.0 |
| 0.1% zVAD-FMK in 25% DMSO + | | | | |
| 75% ethanol | | | | |
| (×0.01 mm) | | | | |
| S.D. | 1.53 | 0.58 | 1.53 | 1.00 |
| Group III | 25.3 | 26.7 | 26.3 | 27.7 |
| 0.1% TAPI-0 in 25% DMSO + | | | | |
| 75% ethanol | | | | |
| (×0.01 mm) | | | | |
| S.D. | 0.58 | 1.15 | 0.58 | 0.58 |
| Group IV | 25.3 | 26.0 | 27.3 | 27.7 |
| 0.1% Tranexamic acid in 25% DMSO + | | | | |
| 75% ethanol | | | | |
| (×0.01 mm) | | | | |
| S.D. | 0.58 | 0.00 | 1.53 | 1.53 |
| Group V | 25.7 | 26.0 | 26.3 | 27.7 |
| 0.1% Soy trypsin inhibitor in 25% DMSO + | | | | |
| 75% ethanol | | | | |
| (×0.01 mm) | | | | |
| S.D. | 0.58 | 0.00 | 1.53 | 1.53 |

RIGHT EAR

Day

| | 0 | 1 | 2 | 3 |
|---|---|---|---|---|
| Group I | 25.7 | 26.0 | 26.7 | 28.0 |
| 25% DMSO + 75% ethanol | | | | |
| (×0.01 mm) | | | | |
| S.D. | 0.58 | 0.00 | 1.15 | 2.00 |
| Group II | 26.7 | 26.7 | 26.3 | 27.0 |
| 25% DMSO + 75% ethanol | | | | |
| (×0.01 mm) | | | | |
| S.D. | 0.58 | 0.58 | 1.53 | 1.00 |
| Group III | 26.0 | 26.3 | 26.3 | 27.7 |
| 25% DMSO + 75% ethanol | | | | |
| (×0.01 mm) | | | | |
| S.D. | 1.00 | 0.58 | 0.58 | 0.58 |
| Group IV | 25.3 | 26.0 | 27.0 | 27.7 |
| 25% DMSO + 75% ethanol | | | | |
| (×0.01 mm) | | | | |
| S.D. | 0.58 | 0.00 | 1.73 | 1.15 |
| Group V | 25.7 | 26.3 | 26.3 | 27.7 |
| 25% DMSO + 75% ethanol | | | | |
| (×0.01 mm) | | | | |
| S.D. | 0.58 | 0.58 | 1.53 | 1.15 |

As seen in Table 5, test sample solutions evaluated did not increase ear thickness when compared to control.

### Example 5

### Effects of Test Samples in Skin

This example shows that test samples including ucf-101 are not irritating to the skin.

The hair of the back skin of mice (FVB strain, female, 7 weeks of age, n=3/group) was clipped by an electric clipper on day 0. 5 μl of test sample solution (˜1 cm diameter) was applied on the back skin. After 24 hours (day 1), skin irritation was measured on the basis of a scoring system (0: no erythema; 1: very slight erythema; 2: well-defined erythema; 3: moderate erythema; 4: several erythema and edema) and a second dose administered. After a second 24 hours (day 2), skin irritation was measured and a third dose administered. After a third 24 hours (day 3), skin irritation was again measured. The results are provided in Table 6.

TABLE 6

Day

| | 0 | 1 | 2 | 3 |
|---|---|---|---|---|
| 0.1% Ucf-101 | 0 | 0 | 0 | 0 |
| in 25% DMSO + 75% ethanol | | | | |
| 0.1% zVAD-FMK | 0 | 0 | 0 | 0 |
| in 25% DMSO + 75% ethanol | | | | |
| 0.1% TAPI-0 | 0 | 0 | 0 | 0 |
| in 25% DMSO + 75% ethanol | | | | |
| 0.1% Tranexamic acid | 0 | 0 | 0 | 0 |
| in 25% DMSO + 75% ethanol | | | | |
| 0.1% Soy trypsin inhibitor | 0 | 0 | 0 | 0 |
| in 25% DMSO + 75% ethanol | | | | |
| 25% DMSO + 75% ethanol | 0 | 0 | 0 | 0 |

As can be seen from Table 6, test sample solutions did not induce skin irritation. These substances were not irritants at a concentration of 0.1%.

### Example 6

### Effect of Omi Inhibition on Wrinkles and Vessel Formation

This example shows that inhibition of Omi/HtrA2 reduces UVB-induced skin damage.

Seven groups of mice (Skh-1 hairless, 7 weeks of age, female, n=3/group) were used: group 1: UVB+0.1% ucf-101 in 25% DMSO+75% ethanol; group 2: UVB+0.1% zVAD−FMK in 25% DMSO+75% ethanol; group 3: UVB+0.1% TAPI-0 in 25% DMSO+75% ethanol; group 4: UVB+0.1% tranexamic acid in 25% DMSO+75% ethanol; group 5: UVB+0.1% soy trypsin inhibitor in 25% DMSO+75% ethanol; group 7: no UVB control.

Groups 1, 2, 3, 4, 5, and 6 were exposed to UVB irradiation using fluorescent lamps (Southern New England Ultraviolet) to deliver 0.35 mW/cm² at the dorsal skin surface of the mice. After irradiation, the indicated samples (100 μl each) for each group were applied to the back skin. This procedure was repeated three times weekly for ten weeks, to simulate chronic UVB exposure. UVB irradiation began with a dose of 0.5 minimum erythema dose (MED) (20 mJ/cm²) and gradually increased in increments of 0.5 MED each day to a maximum dose of 4.5 MED. The total cumulative dose was 6.54 J/cm² UVB.

After 10 weeks, skin wrinkling was evaluated using five categories (0: no wrinkle; 1: slight wrinkle; 2: clear wrinkle; 3: strong wrinkle; 4: severe wrinkle). Mice were sacrificed and back skin samples were snap-frozen in liquid nitrogen. Immunohistochemical staining was performed on 7-micrometer frozen sections using a monoclonal rat anti-mouse CD31 antibody (BD Pharmingen). Representative sections were obtained from UVB irradiated mice and were analyzed using a Nikon E-600 microscope. Images were captured with a SPOT™ digital camera (Diagnostic Instruments), and morphometric analyses were performed using the IP-LAB™ software. Areas occupied by blood vessels were determined in the dermis. Two-sided unpaired Student's t-test was used analyze differences in blood vessels compared to group 1.

The results (mean±S.D.) are shown in Table 7 (wrinkling) and Table 8 (vessel area).

TABLE 7

| Group | Wrinkle category (mean) | S.D. |
|---|---|---|
| 1 | 1.00 | 0.00 |
| 2 | 1.67 | 0.58 |
| 3 | 1.00 | 0.00 |
| 4 | 2.00 | 1.00 |
| 5 | 1.00 | 0.00 |
| 6 | 2.00 | 0.00 |
| 7 | 0.00 | 0.00 |

As seen from Table 7, inhibition of Omi/HtrA2 was effective at preventing and reducing UVB-induced wrinkle formation.

TABLE 8

| | Percent of vessel | | |
|---|---|---|---|
| Group | area (mean) | S.D. | Comparison |
| 1 | 4.7% | 0.85 | — |
| 2 | 8.8% | 0.69 | ** |
| 3 | 8.9% | 1.11 | ** |
| 4 | 8.7% | 2.60 | N.S. |
| 5 | 5.7% | 1.10 | N.S. |
| 6 | 11.1% | 0.40 | *** |
| 7 | 3.3% | 0.42 | N.S. |

** P < 0.01,
*** P < 0.001,
N.S.: No significance

As seen from Table 8, inhibition of Omi/HtrA2 was effective at reducing UVB-induced vessel dilation.

Other embodiments are within the following claims.

1. A method of reducing a wrinkle in a subject, said method comprising administering to the subject a composition comprising an inhibitor of a caspase or serine protease involved in apoptosis in an amount sufficient to reduce said wrinkle.

2. The method of claim 1, wherein the wrinkle is caused by chronic UVB exposure.

3. The method of claim 1, wherein the composition is administered topically.

4. The method of claim 1, wherein the composition is sterile.

5. The method of claim 1, wherein the inhibitor is a caspase inhibitor.

6. The method of claim 1, wherein the inhibitor is an Omi/HtrA2 inhibitor.

7. The method of claim 1, wherein the inhibitor has formula (I): 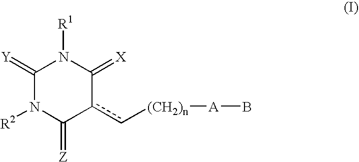
wherein:
Each of R¹ and R² is, independently, aryl, heteroaryl, cycloalkyl, heterocycloalkyl, cycloalkenyl, heterocycloalkenyl, aralkyl, or heteroaralkyl and is optionally substituted with 1-5 R³;
Each of X, Y, and Z is, independently, O or S;
- - - represents an optional double bond;
n is 0-20;
Each of A and B is, independently, aryl or heteroaryl, and is optionally substituted with 1-5 R³; and
Each R³ is, independently, halo, hydroxy, C₁-C₁₀ alkyl, C₁-C₁₀ hydroxyalkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, C₁-C₆ alkylanhydrido, or C₁-C₆ hydroxycarbonylalkyl.

8. The method of claim 7, wherein R¹ and R² are both aryl.

9. The method of claim 8, wherein R¹ and R² are both phenyl.

10. The method of claim 7, wherein one of X, Y, and Z is S, and the other two are O.

11. The method of claim 10, wherein Y is S and X and Z are 0.

12. The method of claim 7, wherein the double bond is present.

13. The method of claim 7, wherein n is 0.

14. The method of claim 7, wherein one of A and B is heteroaryl, and the other is aryl.

15. The method of claim 14, wherein A is heteroaryl and Bis aryl.

16. The method of claim 15, wherein A has formula (II): 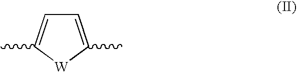
wherein,
W is O, S, or NR^{a}; and
Ra is hydrogen or C₁-C₆ alkyl.

17. The method of claim 16, wherein W is 0.

18. The method of claim 15, wherein B has formula (III): 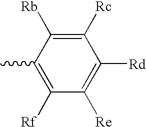
wherein, each of R^{b}, R^{c}, R^{d}, R^{e}, and R^{f} is, independently, hydrogen, halo, hydroxy, C₁-C₁₀ alkyl, C₁-C₁₀ hydroxyalkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, C₁-C₆ alkylanhydrido, or C₁-C₆ hydroxycarbonylalkyl.

19. The method of claim 18, wherein each of R^{b}, R^{c}, R^{d}, R^{e}, and R^{f} is, independently, hydrogen, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

20. The method of claim 18, wherein each of R^{c}, R^{d}, and R^{e} is hydrogen and each of R^{b} and R^{f} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

21. The method of claim 20, wherein one of R^{b} or R^{f} is nitro.

22. The method of claim 18, wherein each of R^{b}, R^{c}, and R^{f} is hydrogen, and each of R^{d} and R^{e} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

23. The method of claim 22, wherein one of R^{d} and R^{e} is halo, and the other is C₁-C₄ alkoxy.

24. The method of claim 23, wherein R^{d} is OCH₃ and R^{e} is C1.

25. The method of claim 18, wherein each of R^{b}, R^{d}, R^{e} and R^{f} is hydrogen, and RC is hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

26. The method of claim 25, wherein R^{c} is carboxy.

27. The method of claim 18, wherein each of R^{c}, R^{d}, and R^{f} is hydrogen, and each of R^{b} and R^{e} is, independently, hydroxy, C₁-C₁₀ alkyl, C₁-C₁₀ alkoxy, C₆-C₁₀ aryl, C₅-C₁₀ heteroaryl, C₃-C₈ heterocyclyl, C₂-C₁₂ alkenyl, C₂-C₁₂ alkynyl, carboxy, carboxylate, nitro, amino, acyl, aminocarbonyl, C₁-C₆ alkylanhydrido, or C₁-C₆ hydroxycarbonylalkyl.

28. The method of claim 27, wherein one of R^{b} and R^{e} is nitro.

29. The method of claim 7, wherein the compound is selected from:

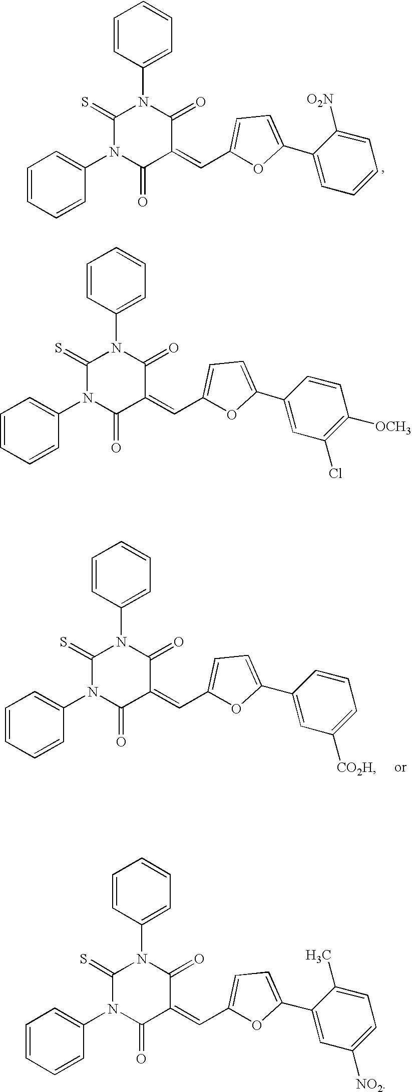

30. The method of claim 7, wherein the wrinkle is caused by exposure to UVB radiation.

31. The method of claim 29, wherein the wrinkle is caused by exposure to UVB radiation.

32. The method of claim 7, wherein the inhibitor is administered topically.

33. The method of claim 29, wherein the inhibitor is administered topically.

34. The method of claim 7, wherein the composition is a cosmetic composition.

35. The method of claim 29, wherein the composition is a cosmetic composition.

36. The method of claim 7, wherein the inhibitor is provided in pharmaceutically suitable carrier.

37. The method of claim 29, wherein the inhibitor is provided in pharmaceutically suitable carrier.

38. The method of claim 7, wherein the composition further comprises a cosmetic agent.

39. The method of claim 29, wherein the composition further comprises a cosmetic agent.

40. A method of providing wrinkle protection to a subject, said method comprising:
supplying to the subject a composition comprising an inhibitor of a caspase or serine protease involved in apoptosis in an amount sufficient to reduce said wrinkle, and
supplying to the subject instructions for using said composition to reduce wrinkles.

41. The method of claim 40, wherein said instructions comprise directions to apply the composition to the skin prior to sun exposure.

42. The method of claim 40, wherein the composition further comprises a cosmetic agent.

43. A composition for topical application, the composition comprising an inhibitor of a caspase or serine protease involved in apoptosis in an amount sufficient to reduce said wrinkle.

44. A method comprising:
identifying a subject in need of reducing a wrinkle
administering an inhibitor of a caspase or serine protease involved in apoptosis in an amount to reduce said wrinkle; and
monitoring the effect of the inhibitor on wrinkle reduction.
